(19) United States
(12) Patent Application Publication
(10) Pub. No.: US 2019/0163270 A1
(43) Pub. Date: May 30, 2019

(54) METHODS AND SYSTEMS FOR FITNESS-MONITORING DEVICE DISPLAYING BIOMETRIC SENSOR DATA-BASED INTERACTIVE APPLICATIONS

(71) Applicant: Fitbit, Inc., San Francisco, CA (US)

(72) Inventors: Robert Louis Da Silva, Fremont, CA (US); Jonathan Wonwook Kim, Emeryville, CA (US); Alexandra Constance Yee, San Francisco, CA (US); Logan Niehaus, Alameda, CA (US); Erin Michelle Leong, Castro Valley, CA (US)

(21) Appl. No.: 16/201,690

(22) Filed: Nov. 27, 2018

Related U.S. Application Data
(60) Provisional application No. 62/591,144, filed on Nov. 27, 2017.

(51) Int. Cl. G06F 3/01; A61B 5/00; A61B 5/024

(52) U.S. Cl. CPC G06F 3/015; G06F 3/017; A61B 2562/0219; A61B 5/744; A61B 5/02438; A61B 5/7275

(57) ABSTRACT

Various embodiments provide a wellness tracking device with integrated electronic components for improving user wellness behaviors in the real world, in which player inputs to an electronic interactive interface element or interactive component are based on sensor data collected via one or more user monitoring devices, the sensor data representing various physical behaviors of the user. In some embodiments, performing certain physical activities or reaching certain wellness goals, as determined by sensors, is required to progress the interactive interface element. In some embodiments, the system is able to determine what a user needs to do (e.g., steps, going to bed) at a certain time in order to reach those wellness goals, and outputs engaging reminders to proactively motivate the user to perform the activities needed to reach the wellness goals.

### CROSS-REFERENCE TO RELATED APPLICATIONS

This application claims priority to U.S. Provisional Patent Application Ser. No. 62/591,144, filed Nov. 27, 2017, and entitled “Gamification Of User Activity Tracking,” which is hereby incorporated herein by reference in its entirety for all purposes.

### BACKGROUND

Wearable electronic devices have gained popularity among consumers. A wearable electronic device may track a user's activities using a variety of sensors. Data captured from these sensors can be analyzed in order to provide a user with information, such as an estimation of how far they walked in a day, their heart rate, how much time they spent sleeping, and the like. Generally, technology is designed to optimize for with data accuracy and speed. However, accurate information alone may not have a meaningful impact to users. The ultimate goal of wearable technology is to help users improve their lifestyle, not just to report it. While it is beneficial for users to have an automated way of tracking some of their behaviors and habits, merely providing users with such direct feedback may not be enough for users to change or improve certain behaviors.

### BRIEF DESCRIPTION OF THE DRAWINGS

Various embodiments in accordance with the present disclosure will be described with reference to the drawings, in which:

FIG. 1 illustrates examples of networked devices that can be used in various embodiments.

FIG. 2 illustrates an example diagram of utilizing user monitoring sensor data as game inputs, in accordance with various embodiments of the present disclosure.

FIG. 3 illustrates an example representation of a function in which game a state or output is based on a comparison between target user behavior and actual user behavior, in accordance with various embodiments of the present disclosure.

FIG. 4A illustrates an example representation of a function in which an avatar mirrors the user behavior, in accordance with various embodiments of the present disclosure.

FIG. 4B illustrates another example representation of a function in which an avatar mirrors the user behavior, in accordance with various embodiments of the present disclosure.

FIG. 5 illustrates an example representation of a function in which multiple user accounts are connected and interactive, in accordance with various embodiments of the present disclosure.

FIG. 6A illustrates a first example of a function in which game progression is based on user behavior detected via a user monitoring device, in accordance with various embodiments of the present disclosure.

FIG. 6B illustrates a second example of a function in which game progression is based on user behavior detected via a user monitoring device, in accordance with various embodiments of the present disclosure.

FIG. 7 illustrates a machine learning model that can be utilized, in accordance with various embodiments of the present disclosure.

FIG. 8 illustrates an example process for determining a state or output based on a comparison between target user behavior and actual user behavior, in accordance with various embodiments of the present disclosure.

FIG. 9 illustrates an example process for determining a game state based on detected user behavior, in accordance with various embodiments of the present disclosure.

FIG. 10 illustrates an example process for determining optimal notification using a trained model, in accordance with various embodiments of the present disclosure.

FIG. 11 illustrates an example process for user activity tracking with an interactive interface, in accordance with various embodiments of the present disclosure.

FIG. 12 illustrates an example process for transforming measured user data into game inputs, in accordance with various embodiments of the present disclosure.

FIG. 13 illustrates a set of basic components of one or more devices of the present disclosure, in accordance with various embodiments of the present disclosure.

### DETAILED DESCRIPTION

In the following description, various embodiments will be described. For purposes of explanation, specific configurations and details are set forth in order to provide a thorough understanding of the embodiments. However, it will also be apparent to one skilled in the art that the embodiments may be practiced without the specific details. Furthermore, well-known features may be omitted or simplified in order not to obscure the embodiment being described.

Systems and methods in accordance with various embodiments of the present disclosure may overcome one or more of the aforementioned and other deficiencies experienced in conventional approaches for electronic wellness tracking. In particular, various embodiments provide an wellness tracking with integrated electronic gaming components for improving user wellness behaviors in the real world, in which player inputs to an sensor-dependent interactive application such as an electronic game or gamification component are based on sensor data collected via one or more user monitoring devices, the sensor data representing various physical behaviors of the user. In some embodiments, performing certain physical activities or reaching certain wellness goals, as determined by sensors, is required to progress the game. In some embodiments, the system is able to determine what a user needs to do (e.g., steps, going to bed) at a certain time in order to reach those wellness goals, and outputs engaging reminders to proactively motivate the user to perform the activities needed to reach the wellness goals.

As individually described in further detail below, the system provides at least the inventive aspects of i) an avatar exhibiting certain characteristics, behaviors, or states based on a difference between a target user behavior during a certain period of time and the actual user behavior as detected by one or more user monitoring devices, in which the avatar characteristic, behavior, or state may represent what the user needs to do to satisfy a certain goal or metric; ii) an avatar whose characteristics, behaviors, or states reflect (i.e., are based on or modified by) in real time, physical behaviors of a user as detected by one or more user monitoring devices; iii) an avatar as a smart companion that provides intelligent and emotionally impactful behavior reminders or suggestions to better assist or guide users in meeting their wellness goals and/or improving wellness behavior, in which the intelligent reminders or suggestions are based at least in part on a user's behavior data as detected via one or more user monitoring devices; iv) a game in which progression or rewards depends on a user accomplishing certain physical tasks or behaviors as detected by one or more user monitoring devices; v) a game or gamification component in which game state is based at least in part on a combination of different types of user behavior data as determined based on various types of biometric data using a plurality of networked devices with different sensor types; and vi) intelligent gamification of user behavior in which various elements of a game adapt to the user based on the user's performance as detected via one or more user monitoring devices, such as to optimally assist the user in meeting wellness goals and improve wellness behaviors, among other aspects described herein. In practice, many of these aspects, among others, may be used in combination.

Various other features and application can be implemented based on, and thus practice, the above described technology and presently disclosed techniques. The present application provides systems and techniques that allow biometric data to be obtained from specific biometric sensors, and transform the biometric data into gaming inputs. In a sense, the present techniques provide a way for computing systems to be controlled biometrically, thereby providing an improvement to the possible functions of computing systems. Yet, such a solution cannot be implemented using conventional devices that lack components such as the specialized electronic devices described herein. Various other applications, processes, and uses are presented below with respect to the various embodiments, which improve various aspects of the operation and performance of the computing device(s) on which they are implemented.

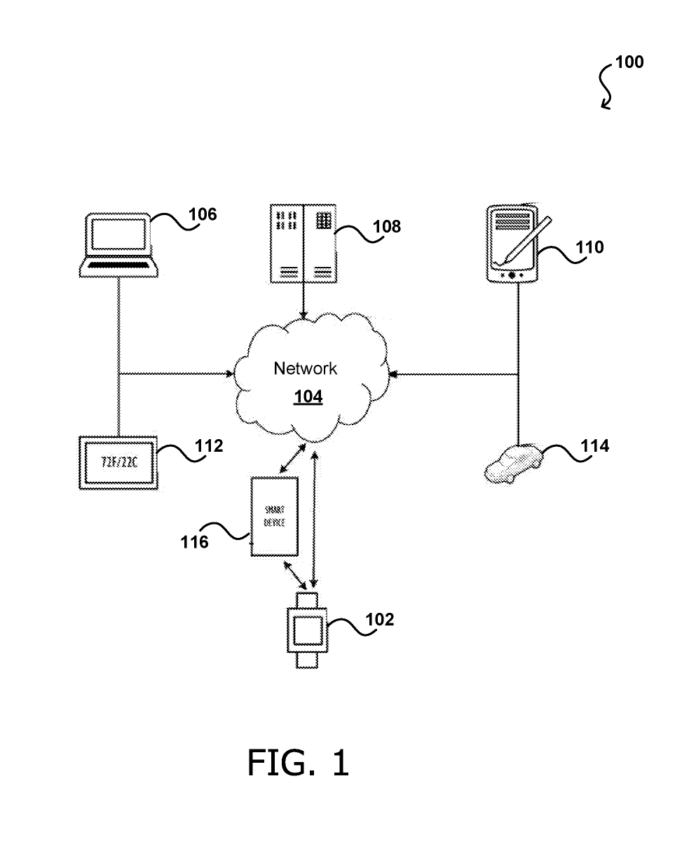

FIG. 1 illustrates examples of networked devices 100 that can be used in accordance with various embodiments. A plurality of devices may be connected to a network 104, enabling communications and data sharing amongst the devices and other devices, databases, servers, etc. For example, a user monitoring device 102 may include wearables such as devices to be worn around the wrist, chest, or other body part, a device to be clipped or otherwise attached onto an article of clothing worn by the user. The user monitoring device 102 may also include a scale, a bed accessory, a tabletop device, among others. The user monitoring device 102 may collectively or respectively capture data related to any one or more of caloric energy expenditure, floors climbed or descended, heart rate, heart rate variability, heart rate recovery, location and/or heading (e.g., through GPS), elevation, ambulatory speed and/or distance traveled, swimming lap count, bicycle distance and/or speed, blood pressure, blood glucose, skin conduction, skin and/or body temperature, electromyography data, electroencephalographic data, weight, body fat, respiration rate and patterns, various body movements, among others. Additional data may be provided from an external source, e.g., the user may input their height, weight, age, stride, or other data in a user profile on a fitness-tracking website or application and such information may be used in combination with some of the above-described data to make certain evaluation or in determining user behaviors, such as the distance traveled or calories burned of the user. The user monitoring devices may also measure or calculate metrics related to the environment around the user such as barometric pressure, weather conditions, light exposure, noise exposure, and magnetic field.

In some embodiments, the user monitoring device 102 may be connected to the network directly, or via an intermediary device 116. For example, the use monitoring device 102 may be connected to the intermediary device 116 via a Bluetooth connection, and the intermediary device 116 may be connected to the network 104 via an Internet connection. In various embodiments, a user may be associated with a user account, and the user account may be associated with (i.e., signed onto) a plurality of different networked devices. In some embodiments, additional devices may provide any of the abovementioned data among other data, and/or receive the data for various processing or analysis. The additional devices may include a computer, a server 108, a handheld device 110, a temperature regulation device 112, or a vehicle 114, among others. Thus, the game state may be determined based on a combination of data collected from these devices.

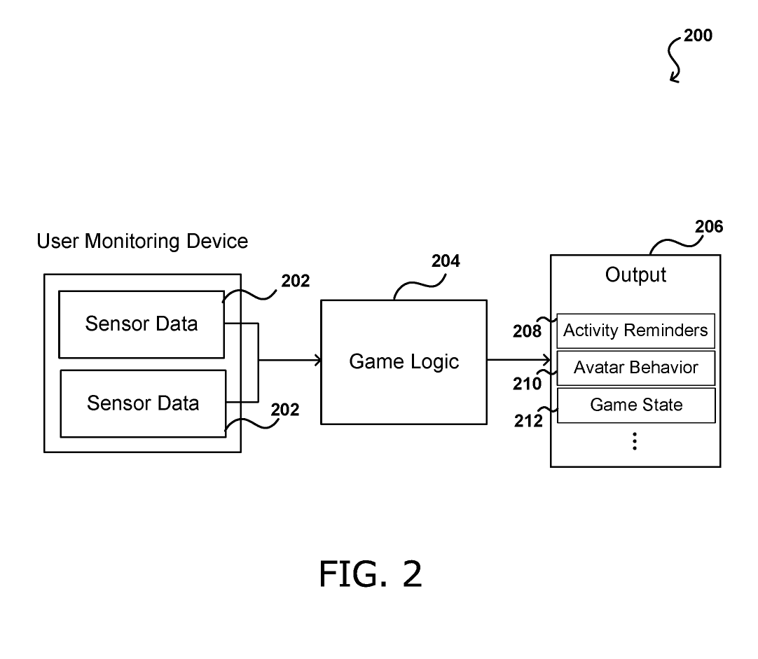

FIG. 2 illustrates an example diagram 200 of utilizing user monitoring sensor data as game inputs, in accordance with various embodiments. The sensor data 202, which may be passively obtained, may be an input into game logic 204 of a game, and used to determine certain outputs 206, including certain reminders 208, avatar behavior 210, or other game states 212. As used herein, “game” may refer to any type of electronic game or gamification component in which player inputs, or the lack thereof, can affect a game state, including modifying a game element such as an avatar (i.e., character, game role, game agent). Game types include, but are not limited to avatar maintenance or evolution type games, progress or adventure type games, task or challenge type games, multi-player (cooperative or competitive) type games, among others. Some games may include elements from more than one game type. The avatar may represent a pet, a character, the device itself, a representation of the user, etc. Avatar behavior may include an action, message, mood, etc., of the avatar as represented in the game. Game state 212 may include a behavior, characteristic, or condition of an avatar, among other aspects of the game, including game levels, game outcomes, game graphics, etc. The biometric data may be derived from data obtained via a suite of various types of sensors, such as those integrated into one or more user monitoring devices. Various embodiments utilize one or more of a wide range of gamification mechanisms to relate detected biometric data of a user with a game state.

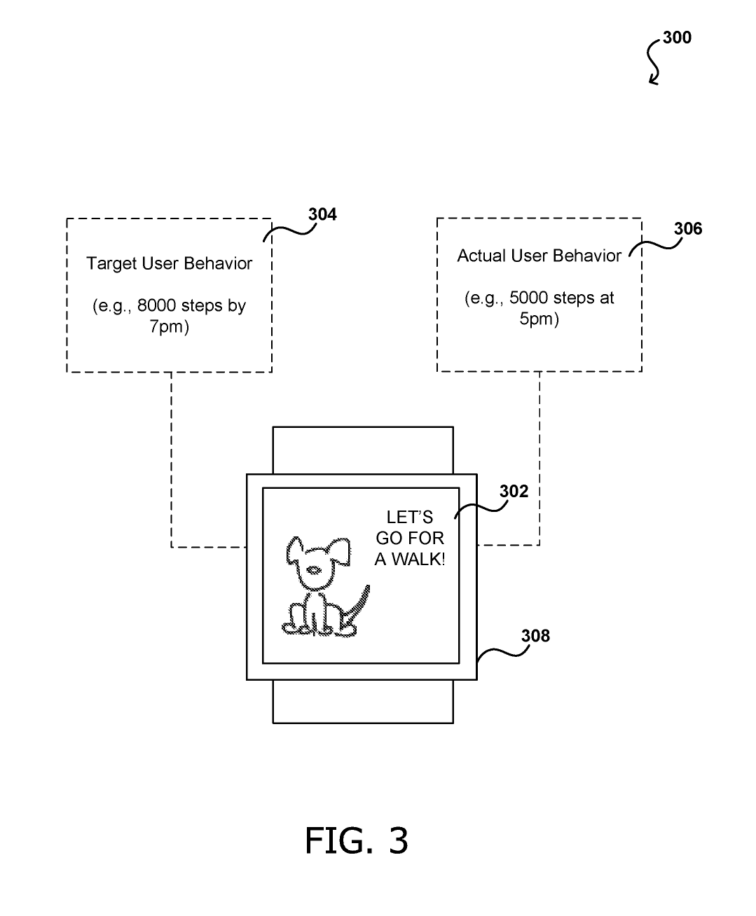

FIG. 3 illustrates an example representation 300 of a function in which game a state 302 or output is based on a comparison between target user behavior 304 and actual user behavior 306, in accordance with various embodiments. In various embodiments, a biometric sensor based gaming approach provides an avatar that exhibits certain characteristics, behaviors, or states based on a difference between a target user behavior 304 during a certain period of time and the actual user behavior 306 as detected by one or more user monitoring devices, in which the avatar characteristic, behavior, or state may represent what the user needs to do to satisfy a certain wellness goal or metric. Thus, this sensor based gamification technique can proactively guide the user to meet their wellness goals, rather than merely measure activity data reactively. For example, a target user behavior 304 may include walking 10,000 steps over the course of a day, and the actual user behavior 306 may be that the user has only walked 6,000 step so far, as detected via a user monitoring device instrumented with pedometry sensors. In this case, the avatar may exhibit a behavior 302 based on the user needing to walk more to reach the target user behavior 304. For example, the avatar may be a dog and the exhibited behavior 302 may be asking to go for a walk, which may be expressed as a output on a user device 308, such as but not necessarily the user monitoring device. The output may include a visual output, such as an animation of the dog holding a leash and wagging its tail, an audio output such as barking sounds, and/or a tactile output such as vibration of the user device. Such a behavior may stop if the user does go for a walk, which can be detected via the user monitoring device. In some embodiments, if the user does go for a walk, the previous behavior of the dog asking to go for a walk may be replaced by a walking behavior, which may be expressed as an animation of the dog happily going on a walk.

In another example, if it is nearing the user's bedtime, which may be a wellness goal previously set by the user, the avatar may exhibit a sleepy state, which may be expressed as an animation of the avatar looking sleepy and/or an audio output of a yawning sound. This serves as a reminder for the user to get ready for bed. Additionally, if it is past the user's bedtime and the user is exhibiting user behavior that indicates the user has not going to bed, the avatar may exhibit a behavior that represents the need for the user to go to sleep. For example, this may be expressed as more petulant avatar behaviors such as nagging the user to go to sleep in order to meet the wellness goal, such as more frequency/louder yawns, wining sounds, vibrations of the user device, and the like. In addition to serving as a reminder as well as appealing to the user's emotions, the behavior of the pet may actually influence the feelings of the user. For example, seeing a visual of the pet snuggling in bed or yawning sounds may actually make the user feel sleepy. Similarly, the avatar may serve as an alarm to wake up the user. For example, appropriate visual, audio, and/or tactile outputs will be generated by the user device. The outputs may persist or increase if it is detected by the user monitoring device that the user still has not gotten out of bed. Additionally, the pet may exhibit energetic behaviors as if it's ready to take on the day, with corresponding visuals and sounds, which may cause the user to feel energized and motivated to get out of bed as well.

Such a sensor based gamification approach may also provide therapeutic guidance. For example, a target user behavior may be to maintain a calm relaxed state. The user monitoring device may detect if a user is stressed or anxious. Thus, the pet may exhibit stressed behavior and provide reminders to the user to calm the pet down by, for example, taking deep breaths and calming themselves. In various embodiments, rather than a cold notification or alarm, the avatar acts as a companion pulling or coaxing the user to perform certain tasks or behaviors by at least in part appealing to the user's emotional reward system. Since the system is capable of knowing whether or not the user has accomplished such tasks or behaviors, the avatar can adjust its behaviors based whether the user is meeting their wellness goals and what the user still needs to do in order to meet certain goals.

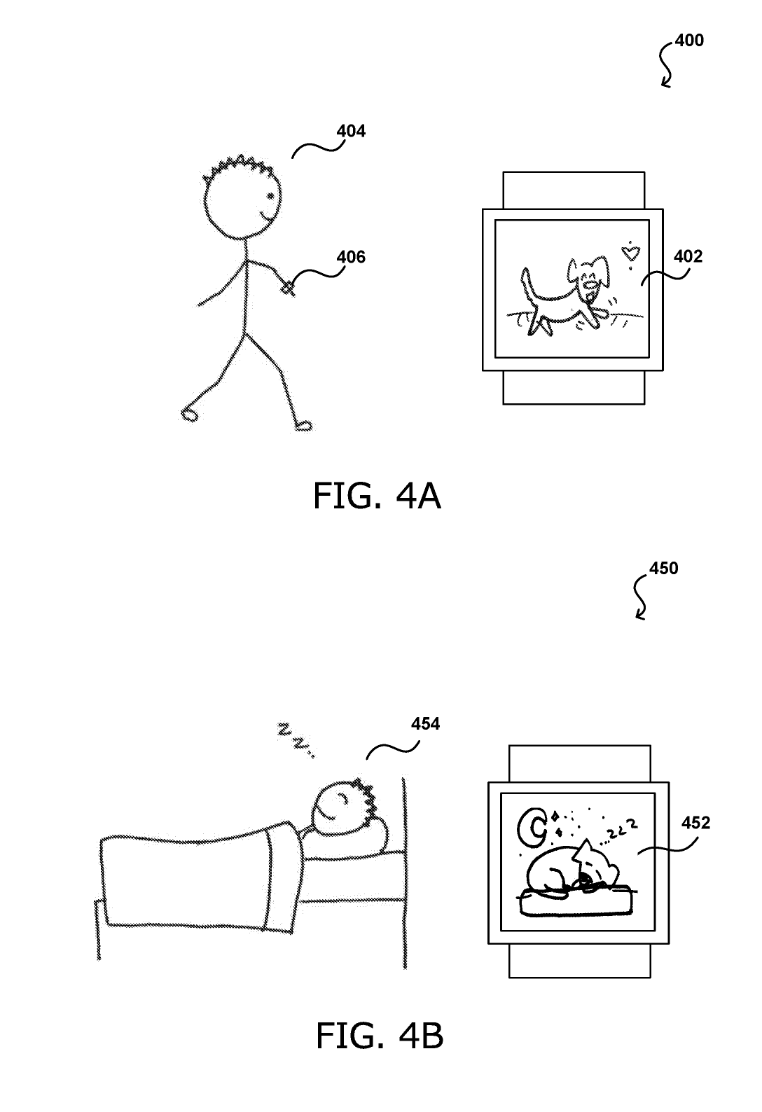

FIGS. 4A and 4B illustrate example representations 400, 450 of a function in which an avatar mirrors the user behavior, in accordance with various embodiments. In various embodiments, a biometric sensor based gaming approach provides an avatar whose characteristics, behaviors, or states reflect, in real time, physical behaviors of a user as detected by one or more user monitoring devices. The avatar characteristics, behaviors, or states may be determined based on detected user behaviors and change when the user behaviors change. For example, as illustrated in FIG. 4A, the avatar 402 may be shown as talking a walk when it is detected that the user 404 is walking. If the user 404 walks faster or begins to run, the avatar 402 may be shown to started running as well, matching the activity of the user 404. If the user 404 is swimming, as detected by the user monitoring device 406, the avatar may be shown as swimming as well. Thus, the avatar 402 serves as an activity companion or “workout buddy”, which may increase the emotional reward of performing such activities.

Similarly, as illustrated in FIG. 4B, the avatar 452 may exhibit a sleep state when it is detected that the user 452 is sleeping. In some embodiments, the avatar 452 may exhibit a working state (e.g., working at a job that is a part of the avatar's backstory) when the user 454 is at work, which may be determined based on activity data and/or location-based data. Additionally, in some embodiments, the avatar 452 may exhibit a stressed state if it is detected that the user 454 is stressed. Thus, the avatar 452 can serve as a reflection of the user 454 and a reminder to make adjustments, such as to attempt to relax or calm down. In some embodiments, the avatar's expressed mood may be based at least in part on the user's behavior as detected by one or more user monitoring devices 406. For example, if the user 454 does not follow a regimen of certain activity or engagement with the avatar 452, the avatar express a negative mood, such as angry, sad, agitated, sleepy, tired, unhappy, or even threaten to run away. Thus, rather than presenting the user 454 with raw data regarding their activity, the data is transformed into an emotionally impactful state.

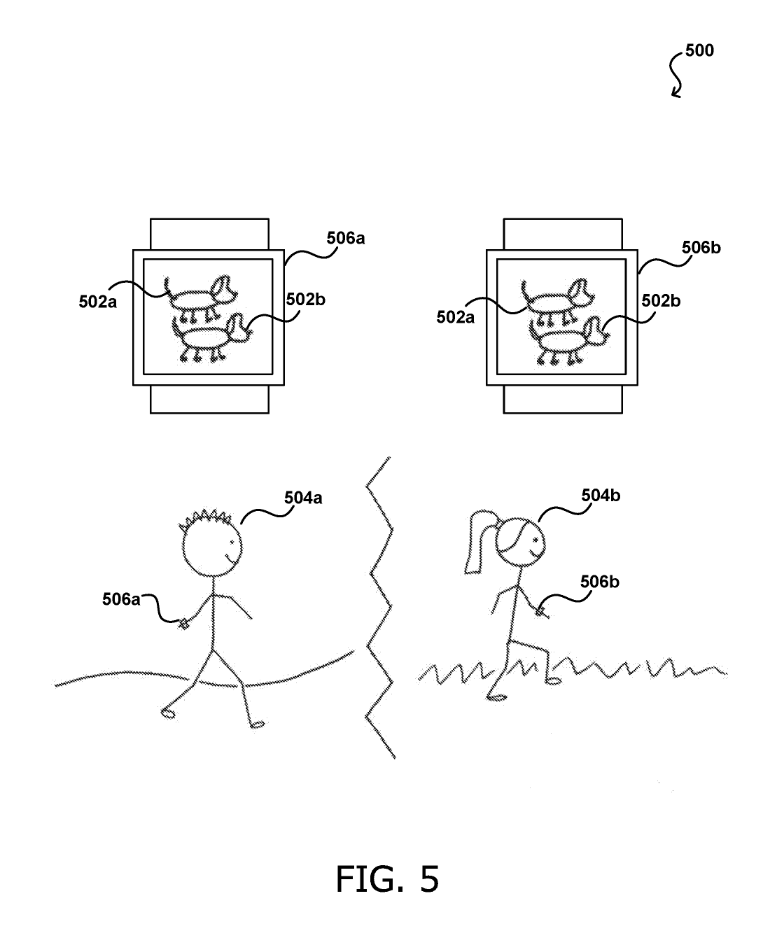

FIG. 5 illustrates an example representation 500 of a function in which multiple user accounts are connected and interactive, in accordance with various embodiments. In some embodiments, avatars 502a, 502b representing different users 504a, 504b can interact together based on the behaviors of the respective users. In an example, such as that illustrated in FIG. 5, a first user 504a may possess a first user monitoring device 506a, on which a first avatar 502a is represented. The first avatar 502a may exhibit certain behaviors and characteristics based on the first user's physical behaviors as detected by the first user monitoring device 506a. A second user 504b may possess a second user monitoring device 506b, on which a second avatar 502b is represented. The second avatar 502b may exhibit certain behaviors and characteristics based on the second user's physical behaviors as detected by the second user monitoring device 506b. In some embodiments, the first user monitoring device 506a and the second user monitoring device 506b may be linked, such that the first avatar 502a may also appear on the second user monitoring device 506b and the second avatar 502b may appear on the first user monitoring device 506a. The first user 504a would be able to see the second user's avatar 502b and the second user 504b would be able to see the first user's avatar 502a. In the example of FIG. 5, the two users 504a, 504b can both go for a walk and both of their user monitoring devices 506a, 506b would show the two avatars 502a, 502b walking together, even if the users 504a, 504b are not at the same place.

In some embodiments, the game may be a competitive type game where two or more players can compete with each other based on certain activity metrics. The competition may be mapped to being a competition between the user's respective pets. An example competition may be a 5 k race. Each of the users could do the run individually, which is detected by the user monitoring device, including the pace time. After all the users in the competition have completed their runs, the race can be simulated within a game with respective avatars, as if the avatars were all racing together, but at the respective pace and completion times in which the users' completed their physical runs. A thirty minute physical run time may be proportionally simulated in two minutes. Thus, these techniques allow users to compete with each other and see a performance comparison, as if they were competing together in real time, while actually performing the competition activities separately on their own time.

These above examples of multi-user interactivity may foster a sense of connectivity between users through their physical activities even if they are not together as well as increases a sense of accountability. Various other strategies may be employed through connecting accounts and enabling interactivity between users within a game setting.

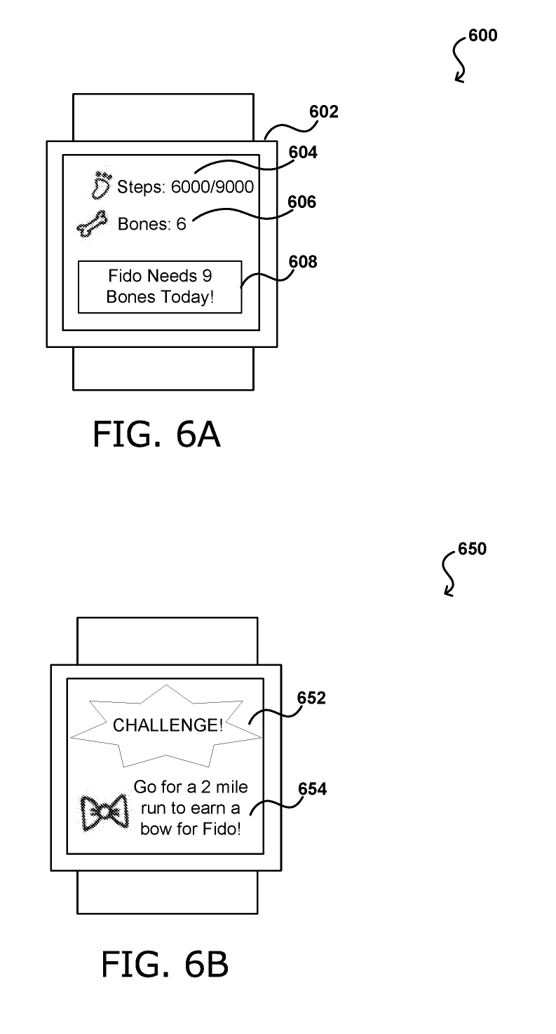

FIGS. 6A and 6B illustrate examples 600, 650 in which game progression is based on user behavior detected via a user monitoring device, in accordance with various embodiments. In various embodiments, a biometric sensor based gaming approach in which game progression, rewards, or certain actions within the game depend on a user accomplishing certain physical tasks or behaviors as detected by one or more user monitoring devices. In some embodiments, there may be maintenance type activities and challenge type activities. A maintenance type activity may be an activity the user needs to perform on a regular basis (e.g., 10,000 steps every day) in order to maintain a certain status in the game. For example, FIG. 6A illustrates a game 602 in which the user is taking care of a pet, and a maintenance activity of 10,000 detected steps may be required every day to feed the pet. Otherwise, the pet may become unhappy and eventually run away. As illustrated in FIG. 6A, the detected number of physical steps 604 taken by the user may translate into an amount of food 606 or other utility within the game that can be used to maintain or advance a game character. There may be status reminders 608 that nudge the user to perform the necessary physical activity to achieve the goal required as a part of the gameplay. FIG. 6B illustrates a challenge type activity, which may be an additional optional activity that the user needs to perform to achieve an additional reward or bonus. For example, a challenge activity to go for a run may provide a prize, such as a treat or an accessory for the pet. As illustrated in FIG. 6B, the game may issue a challenge 652, such as the user going for a two mile run. Upon completion of the run, as detected by a user monitoring device such as a wearable device, a reward 654 (e.g., badge, extra utility) for completing the challenge may be awarded within the game.

In some embodiments, various elements of a game may adapt to the user based on the user's performance as detected via one or more user monitoring devices, such as to optimally assist the user in meeting wellness goals and improve wellness behaviors. For example, the design of the above-described maintenance type activities and challenge type activities may be determined based on the user's fitness level. For a specific user, an initial activity target may be 8,000 steps. Once the user appears to consistently meet this target, the activity target may be raised to 10,000 steps. In certain embodiments, the activity target may be adapted (e.g., raised and lowered) such that it challenges the user but feels attainable such as not to discourage the user. Similarly, challenge type activities may be chosen for a user optimizing for engagement. For example, it may be learned over time that a user is more likely to partake in the challenge when the challenge is issued on a certain day, requires a certain type of activity or activity intensity, among other variables. As described above, various machine learning techniques can be utilized to determine optimal game elements (e.g., maintenance activity requirements and challenges) that best improve wellness behaviors.

In various embodiments, a game or gamification component in which game state, or any of the functions described above, is based at least in part on a combination of different types of user behavior data as determined based on various types of biometric data using a plurality of networked devices with different sensor types. A user may be associated with a user account, and the user account may be associated with (i.e., signed onto) a plurality of different networked devices. For example, the user may have a wearable device that tracks certain activity data, a room monitoring device that tracks environmental data, and other instrumented appliances or devices that are linked to the user account such that data collected from all of these devices are associated with the user account. Thus, the game state may be determined based on a combination of data collected from these devices.

In various embodiments, a biometric sensor-based gaming approach provides an avatar as a smart companion that provides intelligent and emotionally impactful behavior reminders or suggestions to better guide users towards meeting their wellness goals and/or improving their wellness behaviors. As described above, the smart companion can inform the users of what they need to do in order to reach their wellness goals using a variety of different coaxing and motivational mechanisms. The intelligent reminders or suggestions generated at a given time may be determined based at least in part on what a user still needs to do to reach a goal (e.g., target number of steps, target calories burned, bedtime) compared to a user's current state (e.g., number of steps taken so far, calories burned so far, awake status) at that time, as detected via sensors of one or more user monitoring devices. The reminders and suggestions may include visual, audio, and tactile outputs from a user device, which may include one or more user monitoring devices. For example, the user device may be a wearable such as a smart watch, which has a display for displaying a digital image or animation serving as a visual reminder. The wearable may also include and audio output for emitting sounds (e.g., beeps, music, character sounds and speech). The wearable may include a vibration device for generating tactile outputs such as vibrations or “buzzes”.

A user's response to a reminders or suggestion can be recorded and associated with attributes (i.e., metadata) of the reminder or suggestion. An example of a user response include what actions, if any, the user takes after receiving the reminder or suggestion, in which the response is determined based on measurements taken by the sensors of the user monitoring devices. For example, the user response may be whether the user goes to sleep on time after receiving a bedtime reminder. The attributes of the reminder may include, for example, the amount of time between the reminder and the set bedtime, frequency of the reminder, content and format of the reminder (e.g., sleepy pet visuals and sounds, angry pet visuals and sounds, messages, tactile output, no tactile output), among others. The response to a reminder and the attributes of the reminder may form a piece of sample data or training data that, together with other sample data, can be used to determine reminder attributes optimizing for user response. In the above example, the attributes of the bedtime reminder may be optimized for getting the user to go to sleep on time. This technique may be implemented to optimize reminders and suggestions, as well as any other element, for a plurality of target user behaviors.

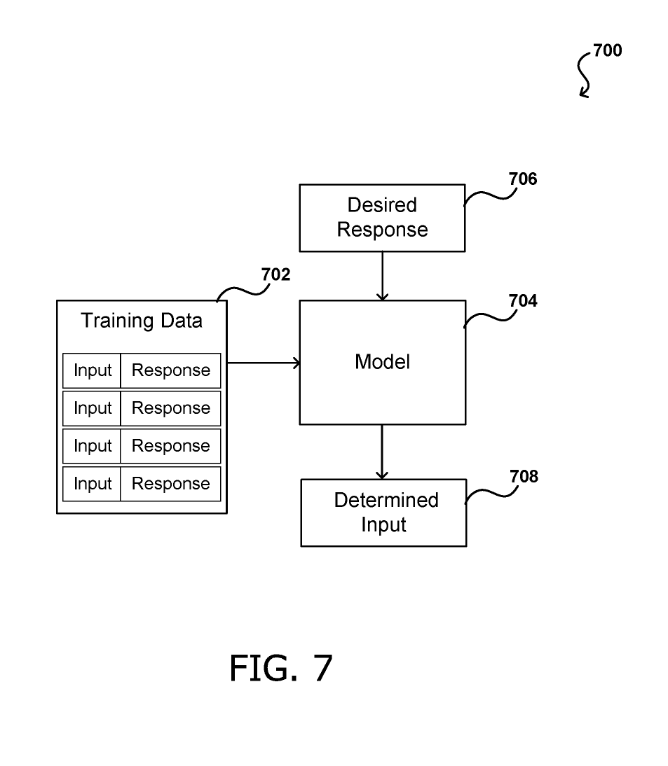

Various machine learning techniques may be utilized to determine optimal reminders and suggestions. FIG. 7 illustrates a machine learning technique 700 that can be utilized, in accordance with various embodiments of the present disclosure. In some embodiments, there may be a vast number of variables and data that determining correlations between user response and reminder attributes (i.e., “inputs”) becomes a mathematically intractable problem. Thus, machine learning techniques may be needed to determine optimal reminders for eliciting a certain user behavior. Example machine learning techniques that can be used include decision tree learning, associated rule learning, artificial neural networks, deep learning, inductive programming logic, support vector machines, clustering, Bayesian networks, reinforcement learning, representation learning, rules based learning, among others or in any combination. Using training data 702 (e.g., data points that represent how a user responded to a certain reminder), a machine learning model 704 can be trained to generate the optimal reminder or suggestion 708, or other output, for eliciting the desired user response 706. In some embodiments, a global model may be utilized, which is a model trained based on data from a pool of users and that can be used to determined optimal reminders for any user. In some embodiments, a local model may be utilized, which is a model generated based on data from a specific user, the model to be used for that user. In certain such embodiments, the local model may also be trained using data from other users but data from the specific user may carry greater weight. The above-described techniques can be used to learn what strategies are able to best motivate users, allowing the system to become increasingly more effective at helping users improve their wellness behaviors.

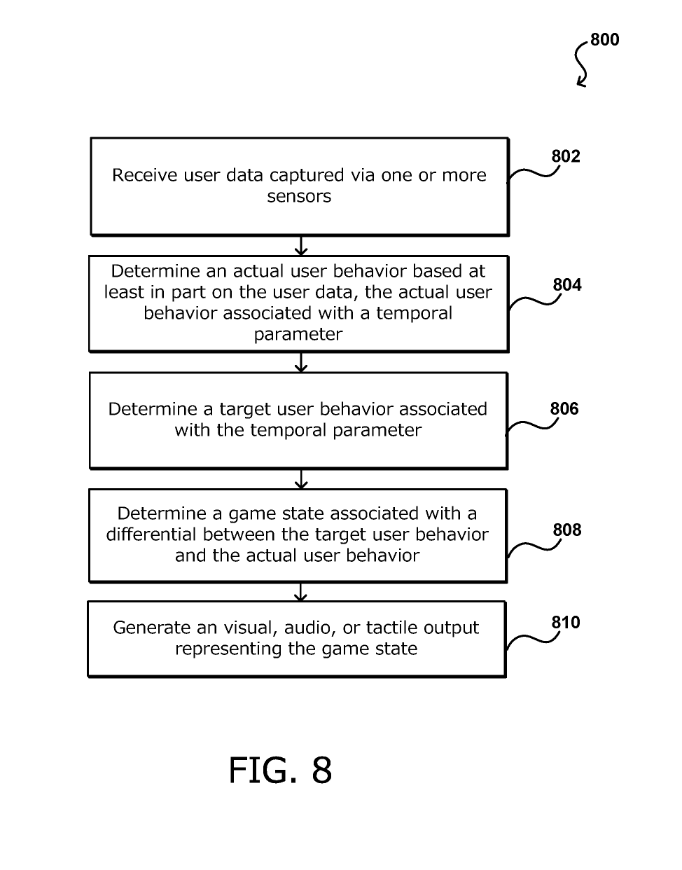

FIG. 8 illustrates an example process 800 for determining a state or output based on a comparison between target user behavior and actual user behavior, in accordance with various embodiments of the present disclosure. It should be understood that, for any process discussed herein, there can be additional, fewer, or alternative steps performed in similar or alternative orders, or in parallel, within the scope of the various embodiments. In this example, user data captured via one or more sensors is received 802, for example at a host server. The one or more sensors may be integrated with a user monitoring devices that is connected to a network, allowing the user data to be received wireless over the network. The user data and the sensors may be any of those described herein, among others. An actual user behavior is then determined 804 based at least in part on the user data, in which the user data is associated with a temporal parameter. In some embodiments, the temporal parameter may refer to any type of time measure, including a time range, such as “the past two hours”, “between 12:00 am and 11:59 pm”, “since Jan. 1, 2017”, etc. A target user behavior associated with the temporal parameter is then determined 806. For example, the target user behavior may be “10,000 steps in 24 hours starting at 12:00 am”, and the actual behavior may be “6,000 steps since 12:00 am”. A game state is determined 808 based on a differential between the target user behavior and the actual user behavior. In this example, the differential may be that 4,000 steps still need to be walked in order for the user to reach the fitness goal of 10,000 steps. An output, such as a visual, audio, or tactile output may be generated 810 representing the game state. For example, the output may include an avatar urging the user to go for a walk.

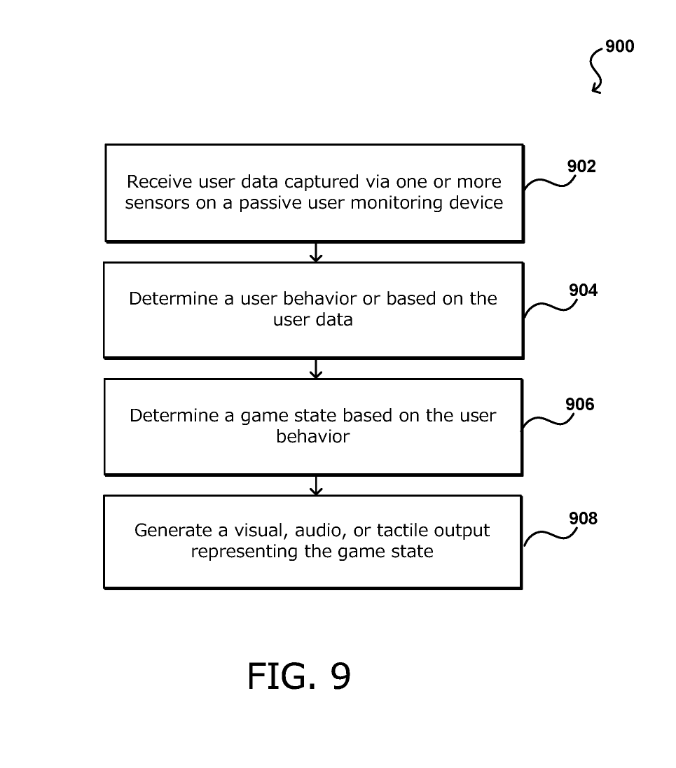

FIG. 9 illustrates an example process 900 for determining a game state based on detected user behavior, in accordance with various embodiments of the present disclosure. In this example, user data captured using one or more sensors on a passive user monitoring device is received 902. A user behavior is then determined 904 based on the user data, and a game state is determined 906 based on the user behavior. For example, the game state may include an avatar behavior, game level, game outcome, etc. An output, such as a visual, audio, or tactile output may be generated 908 representing the game state. For example, the output may be a visual of a pet going for a walk if the determined user behavior indicates that the user is walking.

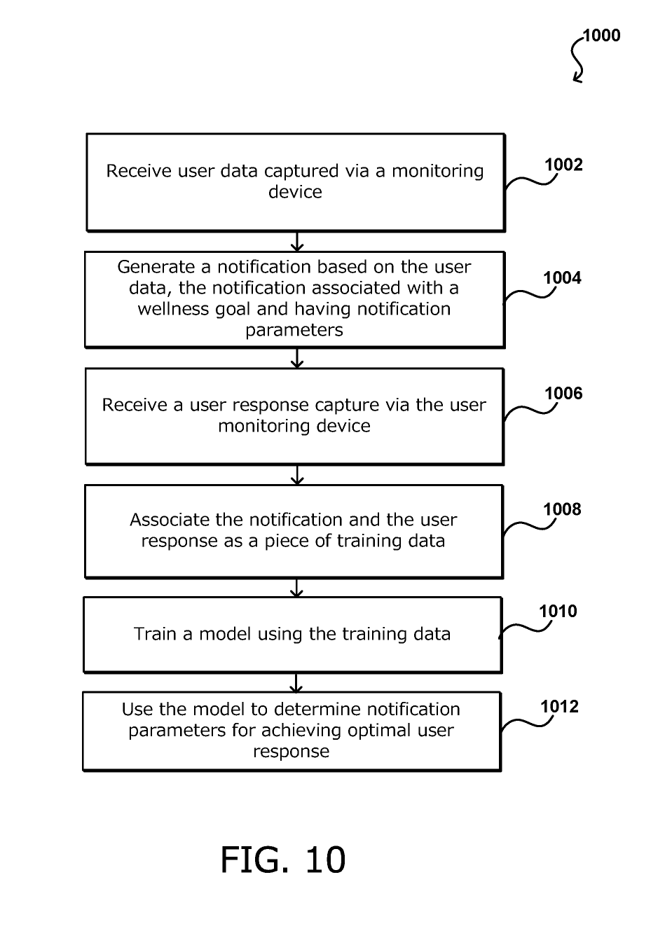

FIG. 10 illustrates an example process 1000 for determining optimal notification using a trained model, in accordance with various embodiments of the present disclosure. In this example, user data capture via a user monitoring device is received 1002. A notification is then generated 1004 based on the user data, in which the notification is associated with a wellness goal and includes notification parameters. For example, this may include a bedtime reminder. The notification can be embodied in various ways, such as represented through avatar actions. The representation and the notification timing relative to the bedtime may be example notification parameters. A user response to the notification is then received 1006 via the user monitoring device. For example, the response may be what time the user actually goes to sleep, as detected via sensors on the user monitoring device. The notification, including its parameters, may be associated 1008 with the user response as a piece of training data. A model can then be trained 1010 using the training data, including addition pairs of notification-response pairs. In some embodiments, parameters of notifications may be adjusted to see how the use responds, and used to train the model. Thus, the model can be used to determine 1012 notification parameters for achieving optimal or desired user response.

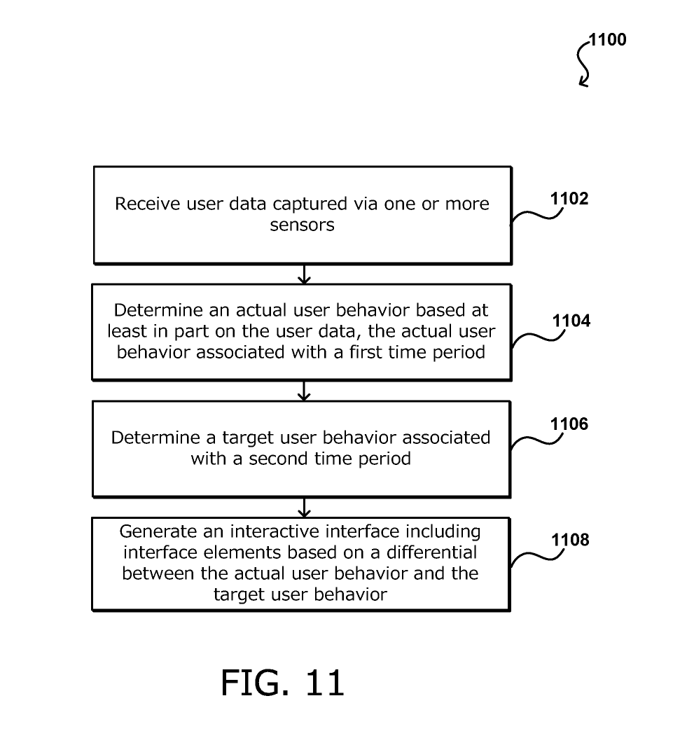

FIG. 11 illustrates an example process 1100 for user activity tracking with dynamic interface, in accordance with various embodiments of the present disclosure. In this example, user data captured via one or more sensors of a user monitoring device is received 1102. The user monitoring device may be worn by a user and provides a virtual sensor-dependent interactive application (e.g., virtual game), in which gameplay of the virtual sensor-dependent interactive application is controlled at least in part based on the captured user data. The use data may include various user biometric or behavior data, such as pulse rate, movement, distance traveled, caloric energy expenditure, heart rate variability, heart rate recovery, location, blood pressure, blood glucose, skin conduction, skin and/or body temperature, electromyography data, electroencephalographic data, sleep status, respiration rate and patterns, or the like. The one or more sensors may include an accelerometer, pulse sensor, microphone, or any combination thereof.

An actual user behavior of the user may be determined 1104 based at least in part on the user data. The actual user behavior may be associated with a previous time period, such as the last 24 hours or since the start of the present day. For example, the actual user behavior may include the number of steps the user has taken since the start of the present day. A target user behavior associated with an upcoming time period may be determined 1106. The target user behavior may be based at least in part on a differential between the actual behavior and a behavior goal. For example, the target user behavior may include a remaining number of steps the user should take before the end of the day to meet the daily goal. In some embodiments, the previous time period and the upcoming time period may or may not overlap. In some embodiments, the second time period is a future time relative to the first time period, or at least include a future time relative to the first time period. For example, the first time period may be the time period between a past starting time and the present time, and the second time period may be the time period between the same past starting time and a future end time.

An interactive interface may be generated 1108 for display, such as on the user monitoring device or a client device separate from the user monitoring device. The interactive interface may include one or more interface elements based at least in part on the target user behavior. In some embodiments, the one or more interface elements includes a graphical representation of an action to be performed in order to achieve the target user behavior based on the actual user behavior. In some embodiments, additional user data captured via the one or more sensors during the upcoming time period may be monitored, and the target behavior and interface elements may be updated based on the additional user data.

In some embodiments, the process 1100 further includes receiving a user profile associated with the user monitoring device, determining the target user behavior based at least in part on the user profile, and updating the user profile based at least in part on the actual user behavior. In some embodiments, the interface elements may include an avatar associated with the user profile, in which a plurality of the visual characteristics of the avatar are determined based at least in part on the user profile. For example, the user profile may include information such as age, weight, height, hair color, etc. and the visual characteristics of the avatar may reflect such information. In some embodiments, the visual characteristics of the interface elements may be updated based on least in part on the user profile and the actual user behavior, in which the user profile data represents a starting point and the actual user behavior represents projected changes to the user profile.

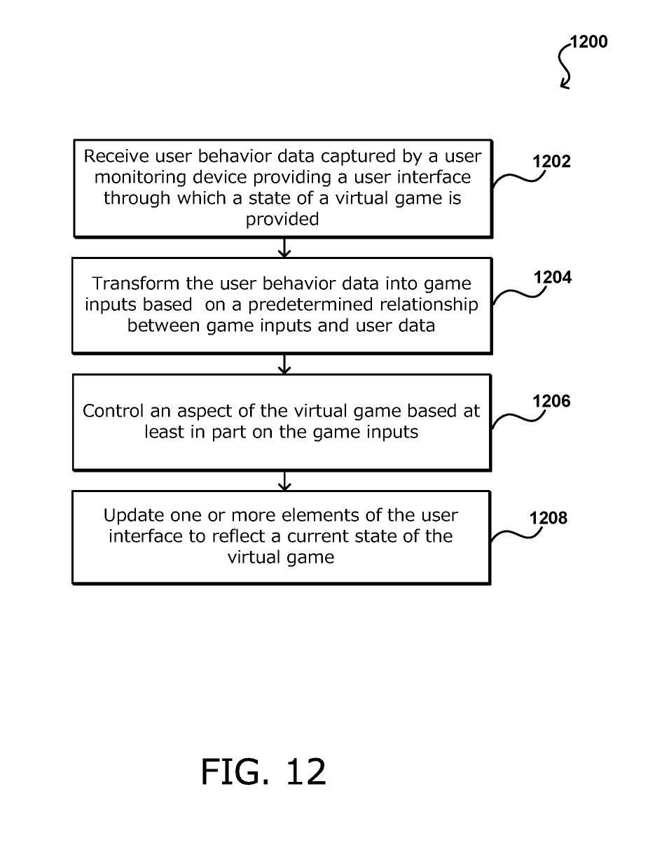

FIG. 12 illustrates an example process 1200 for user activity tracking with dynamic interface, in accordance with various embodiments of the present disclosure. In this example, user data captured via one or more sensors of a user monitoring device is received 1202. The one or more sensors may include at least an accelerometer and a pulse meter and the user data is captured while the user monitoring device is worn by a user for a certain previous time period. The user monitoring device provides a user interface through which a state of a virtual sensor-dependent interactive application is provided. The user data may be used to determine 1204 an activity performed by the user during the previous time period. For example, the user data may indicate that the user was swimming, running, sleeping, or eating, for example. The activity is transformed 1206 into application inputs based at least in part on a predetermined relationship between application inputs and the activity performed by the user. For example, the activity of walking 1000 steps may be transformed into the application input of obtaining food for a virtual pet. Thus, an aspect of the virtual sensor-dependent interactive application may be controlled 1208 based at least in part on the application inputs. One or more elements of the user interface on the user monitoring device may be updated 1210 to reflect a current state of the virtual sensor-dependent interactive application. In some embodiments, the virtual sensor-dependent interactive application includes an avatar exhibiting an avatar behavior, the avatar behavior corresponding to the user activity.

In some embodiments, the virtual sensor-dependent interactive application may include a plurality of possible game levels, and it may be determined that the user behavior data meets one or more conditions for a certain game level. The virtual sensor-dependent interactive application may then be progressed to that game level. In some embodiments, a game goal may be determined based at least in part on the user behavior, and the game goal may be updated based at least in part on changes to the user behavior. Certain game goals or tasks may adapt to the user's fitness level or physical progress. For example, if it is detected that the user typically takes about 5000 steps per day, a game goal may be dynamically set to 6000 steps per day, in order to generate a certain application input. If or when it is detected that the user typically takes about 6000 steps per day, the game goal may be updated to 7000 steps per day. In some embodiments, the game goals may be determined to reflect a goal that pushes the user to improve their behavior, but yet is obtainable. This may cause the use to gradually improve their behavior rather than become discouraged.

In some embodiments, additional user behavior data and/or environmental data may be received. Such data may be measured by one or more other sensors on a second monitoring device or an information source. The additional user behavior data, environment data, or both may also be transformed into additional application inputs. For example, weather information may be received from an information source, and the weather information may be used as an application input. For example, if it is raining, the sensor-dependent interactive application may show that it is raining on the avatar. In some instances, the target user behavior may also adapt to such information. Instead of going for a walk, the target use behavior may change to stretching or other indoor activity.

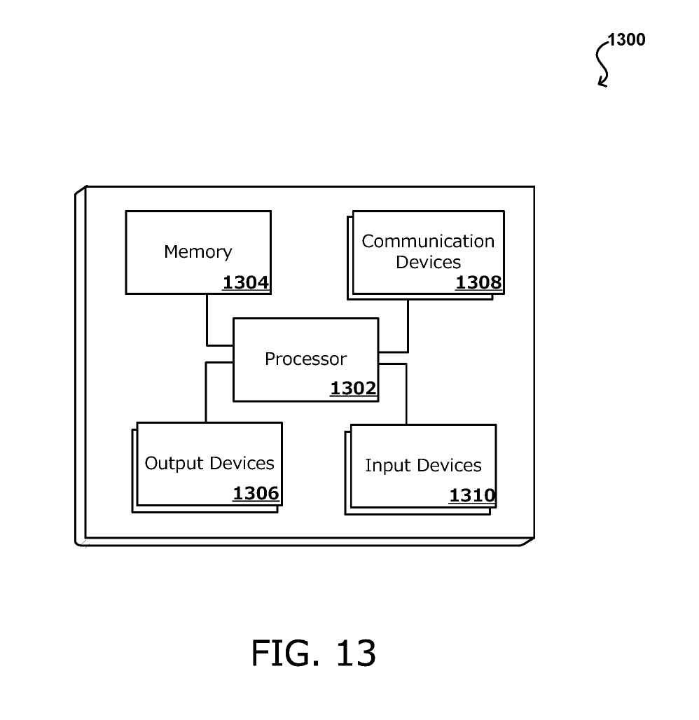

FIG. 13 illustrates a set of basic components 1300 of one or more devices of the present disclosure, in accordance with various embodiments of the present disclosure. In this example, the device includes at least one processor 1302 for executing instructions that can be stored in a memory device or element 1304. As would be apparent to one of ordinary skill in the art, the device can include many types of memory, data storage or computer-readable media, such as a first data storage for program instructions for execution by the at least one processor 1302, the same or separate storage can be used for images or data, a removable memory can be available for sharing information with other devices, and any number of communication approaches can be available for sharing with other devices. The device may include at least one type of output device 1306, such as a touch screen, electronic ink (e-ink), organic light emitting diode (OLED) or liquid crystal display (LCD), although devices such as servers might convey information via other means, such as through a system of lights and data transmissions. The device typically will include one or more networking device 1308, such as a port, network interface card, or wireless transceiver that enables communication over at least one network. The device can include at least one input device 1310 able to receive conventional input from a user. This conventional input can include, for example, a push button, touch pad, touch screen, wheel, joystick, keyboard, mouse, trackball, keypad or any other such device or element whereby a user can input a command to the device. These I/O devices could even be connected by a wireless infrared or Bluetooth or other link as well in some embodiments. In some embodiments, however, such a device might not include any buttons at all and might be controlled only through a combination of visual and audio commands such that a user can control the device without having to be in contact with the device.

As discussed, different approaches can be implemented in various environments in accordance with the described embodiments. As will be appreciated, although a Web-based environment is used for purposes of explanation in several examples presented herein, different environments may be used, as appropriate, to implement various embodiments. The system includes an electronic client device, which can include any appropriate device operable to send and receive requests, messages or information over an appropriate network and convey information back to a user of the device. Examples of such client devices include personal computers, cell phones, handheld messaging devices, laptop computers, set-top boxes, personal data assistants, electronic book readers and the like. The network can include any appropriate network, including an intranet, the Internet, a cellular network, a local area network or any other such network or combination thereof. Components used for such a system can depend at least in part upon the type of network and/or environment selected. Protocols and components for communicating via such a network are well known and will not be discussed herein in detail. Communication over the network can be enabled via wired or wireless connections and combinations thereof. In this example, the network includes the Internet, as the environment includes a Web server for receiving requests and serving content in response thereto, although for other networks, an alternative device serving a similar purpose could be used, as would be apparent to one of ordinary skill in the art.

The illustrative environment includes at least one application server and a data store. It should be understood that there can be several application servers, layers or other elements, processes or components, which may be chained or otherwise configured, which can interact to perform tasks such as obtaining data from an appropriate data store. As used herein, the term “data store” refers to any device or combination of devices capable of storing, accessing and retrieving data, which may include any combination and number of data servers, databases, data storage devices and data storage media, in any standard, distributed or clustered environment. The application server can include any appropriate hardware and software for integrating with the data store as needed to execute aspects of one or more applications for the client device and handling a majority of the data access and business logic for an application.

The application server provides access control services in cooperation with the data store and is able to generate content such as text, graphics, audio and/or video to be transferred to the user, which may be served to the user by the Web server in the form of HTML, XML or another appropriate structured language in this example. The handling of all requests and responses, as well as the delivery of content between the client device and the application server, can be handled by the Web server. It should be understood that the Web and application servers are not required and are merely example components, as structured code discussed herein can be executed on any appropriate device or host machine as discussed elsewhere herein. The data store can include several separate data tables, databases or other data storage mechanisms and media for storing data relating to a particular aspect. For example, the data store illustrated includes mechanisms for storing content (e.g., production data) and user information, which can be used to serve content for the production side. The data store is also shown to include a mechanism for storing log or session data. It should be understood that there can be many other aspects that may need to be stored in the data store, such as page image information and access rights information, which can be stored in any of the above listed mechanisms as appropriate or in additional mechanisms in the data store. The data store is operable, through logic associated therewith, to receive instructions from the application server and obtain, update or otherwise process data in response thereto. In one example, a user might submit a search request for a certain type of item. In this case, the data store might access the user information to verify the identity of the user and can access the catalog detail information to obtain information about items of that type. The information can then be returned to the user, such as in a results listing on a Web page that the user is able to view via a browser on the user device. Information for a particular item of interest can be viewed in a dedicated page or window of the browser.

Each server typically will include an operating system that provides executable program instructions for the general administration and operation of that server and typically will include computer-readable medium storing instructions that, when executed by a processor of the server, allow the server to perform its intended functions. Suitable implementations for the operating system and general functionality of the servers are known or commercially available and are readily implemented by persons having ordinary skill in the art, particularly in light of the disclosure herein.

The environment in one embodiment is a distributed computing environment utilizing several computer systems and components that are interconnected via communication links, using one or more computer networks or direct connections. However, it will be appreciated by those of ordinary skill in the art that such a system could operate equally well in a system having fewer or a greater number of components than are illustrated. Thus, the depiction of the systems herein should be taken as being illustrative in nature and not limiting to the scope of the disclosure.

The various embodiments can be further implemented in a wide variety of operating environments, which in some cases can include one or more user computers or computing devices which can be used to operate any of a number of applications. User or client devices can include any of a number of general purpose personal computers, such as desktop or notebook computers running a standard operating system, as well as cellular, wireless and handheld devices running mobile software and capable of supporting a number of networking and messaging protocols. Devices capable of generating events or requests can also include wearable computers (e.g., smart watches or glasses), VR headsets, Internet of Things (IoT) devices, voice command recognition systems, and the like. Such a system can also include a number of workstations running any of a variety of commercially-available operating systems and other known applications for purposes such as development and database management. These devices can also include other electronic devices, such as dummy terminals, thin-clients, gaming systems and other devices capable of communicating via a network.

Most embodiments utilize at least one network that would be familiar to those skilled in the art for supporting communications using any of a variety of commercially-available protocols, such as TCP/IP, FTP, UPnP, NFS, and CIFS. The network can be, for example, a local area network, a wide-area network, a virtual private network, the Internet, an intranet, an extranet, a public switched telephone network, an infrared network, a wireless network and any combination thereof.

In embodiments utilizing a Web server, the Web server can run any of a variety of server or mid-tier applications, including HTTP servers, FTP servers, CGI servers, data servers, Java servers and business application servers. The server(s) may also be capable of executing programs or scripts in response requests from user devices, such as by executing one or more Web applications that may be implemented as one or more scripts or programs written in any programming language, such as Java®, C, C# or C++ or any scripting language, such as Perl, Python or TCL, as well as combinations thereof. The server(s) may also include database servers, including without limitation those commercially available from Oracle®, Microsoft®, Sybase® and IBM® as well as open-source servers such as MySQL, Postgres, SQLite, MongoDB, and any other server capable of storing, retrieving and accessing structured or unstructured data. Database servers may include table-based servers, document-based servers, unstructured servers, relational servers, non-relational servers or combinations of these and/or other database servers.

The environment can include a variety of data stores and other memory and storage media as discussed above. These can reside in a variety of locations, such as on a storage medium local to (and/or resident in) one or more of the computers or remote from any or all of the computers across the network. In a particular set of embodiments, the information may reside in a storage-area network (SAN) familiar to those skilled in the art. Similarly, any necessary files for performing the functions attributed to the computers, servers or other network devices may be stored locally and/or remotely, as appropriate. Where a system includes computerized devices, each such device can include hardware elements that may be electrically coupled via a bus, the elements including, for example, at least one central processing unit (CPU), at least one input device (e.g., a mouse, keyboard, controller, touch-sensitive display element or keypad) and at least one output device (e.g., a display device, printer or speaker). Such a system may also include one or more storage devices, such as disk drives, optical storage devices and solid-state storage devices such as random access memory (RAM) or read-only memory (ROM), as well as removable media devices, memory cards, flash cards, etc.

Such devices can also include a computer-readable storage media reader, a communications device (e.g., a modem, a network card (wireless or wired), an infrared communication device) and working memory as described above. The computer-readable storage media reader can be connected with, or configured to receive, a computer-readable storage medium representing remote, local, fixed and/or removable storage devices as well as storage media for temporarily and/or more permanently containing, storing, transmitting and retrieving computer-readable information. The system and various devices also typically will include a number of software applications, modules, services or other elements located within at least one working memory device, including an operating system and application programs such as a client application or Web browser. It should be appreciated that alternate embodiments may have numerous variations from that described above. For example, customized hardware might also be used and/or particular elements might be implemented in hardware, software (including portable software, such as applets) or both. Further, connection to other computing devices such as network input/output devices may be employed.

Storage media and other non-transitory computer readable media for containing code, or portions of code, can include any appropriate media known or used in the art, such as but not limited to volatile and non-volatile, removable and non-removable media implemented in any method or technology for storage of information such as computer readable instructions, data structures, program modules or other data, including RAM, ROM, EEPROM, flash memory or other memory technology, CD-ROM, digital versatile disk (DVD) or other optical storage, magnetic cassettes, magnetic tape, magnetic disk storage or other magnetic storage devices or any other medium which can be used to store the desired information and which can be accessed by a system device. Based on the disclosure and teachings provided herein, a person of ordinary skill in the art will appreciate other ways and/or methods to implement the various embodiments.

While various embodiments of the invention have been described above, it should be understood that they have been presented by way of example only, and not by way of limitation. Likewise, the various diagrams may depict an example architectural or other configuration for the disclosure, which is done to aid in understanding the features and functionality that can be included in the disclosure. The disclosure is not restricted to the illustrated example architectures or configurations, but can be implemented using a variety of alternative architectures and configurations. Additionally, although the disclosure is described above in terms of various exemplary embodiments and implementations, it should be understood that the various features and functionality described in one or more of the individual embodiments are not limited in their applicability to the particular embodiment with which they are described. They instead can be applied, alone or in some combination, to one or more of the other embodiments of the disclosure, whether or not such embodiments are described, and whether or not such features are presented as being a part of a described embodiment. Thus the breadth and scope of the present disclosure should not be limited by any of the above-described exemplary embodiments.

Unless otherwise defined, all terms (including technical and scientific terms) are to be given their ordinary and customary meaning to a person of ordinary skill in the art, and are not to be limited to a special or customized meaning unless expressly so defined herein. It should be noted that the use of particular terminology when describing certain features or aspects of the disclosure should not be taken to imply that the terminology is being re-defined herein to be restricted to include any specific characteristics of the features or aspects of the disclosure with which that terminology is associated. Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term includes' should be interpreted as ‘includes but is not limited to;’ the term ‘example’ is used to provide exemplary instances of the item in discussion, not an exhaustive or limiting list thereof; adjectives such as ‘known’, ‘normal’, ‘standard’, and terms of similar meaning should not be construed as limiting the item described to a given time period or to an item available as of a given time, but instead should be read to encompass known, normal, or standard technologies that may be available or known now or at any time in the future; and use of terms like ‘preferably,’ ‘preferred,’ ‘desired,’ or ‘desirable,’ and words of similar meaning should not be understood as implying that certain features are critical, essential, or even important to the structure or function of the invention, but instead as merely intended to highlight alternative or additional features that may or may not be utilized in a particular embodiment of the invention. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

Where a range of values is provided, it is understood that the upper and lower limit, and each intervening value between the upper and lower limit of the range is encompassed within the embodiments.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” does not exclude a plurality. A single processor or other unit may fulfill the functions of several items recited in the claims. The mere fact that certain measures are recited in mutually different dependent claims does not indicate that a combination of these measures cannot be used to advantage. Any reference signs in the claims should not be construed as limiting the scope.

It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

All numbers expressing quantities of ingredients, reaction conditions, and so forth used in the specification are to be understood as being modified in all instances by the term ‘about.’ Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

All of the features disclosed in this specification (including any accompanying exhibits, claims, abstract and drawings), and/or all of the steps of any method or process so disclosed, may be combined in any combination, except combinations where at least some of such features and/or steps are mutually exclusive. The disclosure is not restricted to the details of any foregoing embodiments. The disclosure extends to any novel one, or any novel combination, of the features disclosed in this specification (including any accompanying claims, abstract and drawings), or to any novel one, or any novel combination, of the steps of any method or process so disclosed.

Various modifications to the implementations described in this disclosure may be readily apparent to those skilled in the art, and the generic principles defined herein may be applied to other implementations without departing from the spirit or scope of this disclosure. Thus, the disclosure is not intended to be limited to the implementations shown herein, but is to be accorded the widest scope consistent with the principles and features disclosed herein. Certain embodiments of the disclosure are encompassed in the claim set listed below or presented in the future.

What is claimed is:

1. A computer-implemented method, comprising:
receiving user data captured via one or more sensors of a user monitoring device worn by a user, the user monitoring device providing a virtual sensor-dependent interactive application, wherein at least one aspect of the virtual sensor-dependent interactive application is determined at least in part based on the captured user data;
determining an actual user behavior based at least in part on the user data, the actual user behavior associated with a previous time period;
determining a target user behavior for an upcoming time period based at least in part on a comparison between the actual behavior and a behavior goal; and
generating an interactive interface for display on the user monitoring device, the interactive interface including one or more interface elements based at least in part on the target user behavior.

2. The method of claim 1, further comprising:
monitoring additional user data captured via the one or more sensors during the upcoming time period; and
updating the target behavior and interface elements based on the additional user data.

3. The method of claim 1, wherein the one or more interface elements includes a graphical representation of an action to be performed in order to achieve the target user behavior based on the actual user behavior.

4. The method of claim 1, further comprising:
receiving a user profile associated with the user monitoring device;
determining the target user behavior based at least in part on the user profile; and
updating the user profile based at least in part on the actual user behavior.

5. The method of claim 1, further comprising:
receiving a user profile associated with the user monitoring device;
generating an avatar associated with the user profile; and
determining a plurality of visual characteristics of the avatar based at least in part on the user profile.

6. The method of claim 5, further comprising:
updating one or more of the plurality of visual characteristics based on least in part on the actual user behavior, wherein the user profile data represents a starting point and the actual user behavior represents projected changes to the user profile.

7. A user monitoring device, comprising:
one or more sensors, including at least an accelerometer and a pulse meter;
a user interface;
at least one processor; and
non-transitory computer-readable memory including instructions that, when executed by the at least one processor, cause the system to:
provide a virtual sensor-dependent interactive application;
measure user data via the one or more sensors while the user monitoring device is worn by a user, the user behavior data associated with a previous time period;
determine an activity performed by the user during the previous time period based on the user data;
transform the activity into application inputs based at least in part on a predetermined relationship between application inputs and activities performed by the user;
control an aspect of the virtual sensor-dependent interactive application based at least in part on the application inputs; and
update one or more elements of the user interface on the user monitoring device to reflect a current state of the virtual sensor-dependent interactive application.

8. The device of claim 7, wherein the non-transitory computer-readable memory includes instructions that, when executed by the at least one processor, further cause the system to:
determine that the user activity meets one or more conditions for an application level, wherein the virtual sensor-dependent interactive application includes a plurality of possible application levels; and
progress the virtual gave to the application level.

9. The device of claim 7, wherein the non-transitory computer-readable memory includes instructions that, when executed by the at least one processor, further cause the system to:
determine an application goal based at least in part on the user activity; and
update the application goal based at least in part on changes to the user activity.

10. The device of claim 7, wherein the virtual sensor-dependent interactive application includes an avatar exhibiting an avatar behavior, the avatar behavior corresponding to the user activity.

11. The device of claim 7, wherein the non-transitory computer-readable memory includes instructions that, when executed by the at least one processor, further cause the system to:
receive additional user data, environment data, or both, measured by one or more other sensors on a second monitoring device; and
transform the additional user data, environment data, or both into additional application inputs.

12. The device of claim 7, wherein the user data includes one or more of: pulse rate, movement, distance traveled, caloric energy expenditure, heart rate variability, heart rate recovery, location, blood pressure, blood glucose, skin conduction, skin and/or body temperature, electromyography data, electroencephalographic data, or respiration rate and patterns.

13. The device of claim 7, wherein the one or more sensors include at least one of a pulse meter, oximeter, accelerometer, location device, temperature sensor, pedometry sensor, or microphone.

14. A computer-implemented method, comprising:
receiving user data captured via one or more sensors of a user monitoring device worn by a user during a previous time period, the user monitoring device providing a user interface through which a state of a virtual sensor-dependent interactive application is provided;
determining an activity performed by the user during the previous time period based on the user data;
transforming the user activity into application inputs based at least in part on a predetermined relationship between application inputs and user activities;
controlling an aspect of the virtual sensor-dependent interactive application based at least in part on the application inputs; and
updating one or more elements of the user interface on the user monitoring device to reflect a current state of the virtual sensor-dependent interactive application.

15. The method of claim 14, further comprising:
determining that the user activity meets one or more conditions for an application level, wherein the virtual sensor-dependent interactive application includes a plurality of possible application levels; and
progressing the virtual gave to the application level.

16. The method of claim 14, further comprising:
determining an application goal based at least in part on the user activity; and
updating the application goal based at least in part on changes to the user activity.

17. The method of claim 14, wherein the virtual sensor-dependent interactive application includes an avatar exhibiting an avatar behavior, the avatar behavior corresponding to the user activity.

18. The method of claim 14, further comprising:
receiving additional user data, environment data, or both, measured by one or more other sensors on a second monitoring device; and
transforming the additional user data, environment data, or both into additional application inputs.

19. The method of claim 14, wherein the user data includes one or more of: pulse rate, movement, distance traveled, caloric energy expenditure, heart rate variability, heart rate recovery, location, blood pressure, blood glucose, skin conduction, skin and/or body temperature, electromyography data, electroencephalographic data, or respiration rate and patterns.

20. The method of claim 14, wherein the one or more sensors include at least one of a pulse meter, oximeter, accelerometer, location device, temperature sensor, pedometry sensor, or microphone.
